(19) United States
(12) Patent Application Publication
(10) Pub. No.: US 2010/0061945 A1
(43) Pub. Date: Mar. 11, 2010

(54) Use Of Quinoa Extract As Cosmetic And Pharmaceutic Slimming Agent And/Or As An Agent Preventing The Formation Of New Fats In The Human Body

(75) Inventors: Christine Garcia, Castres (FR); Corinne Stoltz, Thiais (FR)

Correspondence Address: AIR LIQUIDE;Intellectual Property, 2700 POST OAK BOULEVARD, SUITE 1800, HOUSTON, TX, 77056, US

(73) Assignee: Societe D'Exploitation Des Produits Pour Les Indus tries Chimiques Seppic, Paris (FR)

(21) Appl. No.: 12/441,644

(22) PCT Filed: Aug. 31, 2007
(86) PCT No.: PCT/FR07/51859

§ 371 Date: Mar. 17, 2009

(30) Foreign Application Priority Data
Sep. 18, 2006 (FR) 0653801
Sep. 18, 2006 (FR) 0654790

(51) Int. Cl. A61K 8/97; A61Q 17/04; A61Q 19/06

(52) U.S. Cl. 424/59; 424/725

(57) ABSTRACT

The invention relates to the use of Quinoa extract as a slimming agent and/or as an agent preventing the formation of new fats in the body, in a composition comprising a cosmetically acceptable medium. The invention relates to a non-therapeutic treatment method using said extract and to the use of said extract for preparing a drug with lipolytic activity for inducing a slimming effect on the human body and for preparing a drug for preventing the formation of new fats in the human body.

The subject of the present invention is a novel use of cosmetic active agents for slimming the human body and/or for preventing the formation of fat in the human body.

Some of the fat in the human body is stored in the form of triglycerides, in cells of the fatty tissue of the dermis, called adipocytes. Slimming reflects a reduction in the fat stored in the adipocytes. This process requires a preliminary step which takes place inside these cells and which consists in hydrolyzing the triglycerides to fatty acids and glycerol. This phenomenon is called lipolysis. Most slimming cosmetic formulations currently marketed contain at least one compound possessing a lipolytic activity. The one most frequently used is caffeine, but theophylline is also known to possess such a property.

By contrast, during weight gain, the adipocytes can fill with fat in the context of a so-called adipocyte atrophy process but a phenomenon of recruitment of new adipocytes by differentiation of preadipocytes to mature adipocytes, capable of storing the triglycerides according to the adipocyte hyperplasia process, can also occur. An active agent which makes it possible to prevent weight gain can therefore be selected based on its ability to prevent the recruitment of new adipocytes. Tumor necrosis factor or TNFα is widely described in the scientific literature as an inhibitor of this maturation of the adipocytes but its use is not allowed in cosmetic formulations. No active molecule therefore exists that is known for its use in cosmetics in the state of the art and which constitutes a reference inhibitor of the differentiation of preadipocytes.

During their search for novel active agents with lipolytic activity, and/or which make it possible to inhibit the differentiation of the preadipocytes in order to prevent the formation of new fat, which have good compatibility with the skin, the inventors demonstrated that quinoa extracts, which are normally marketed by the cosmetics industries for their moisturizing and decongestant properties, had both a lipolytic effect that is more effective than that of caffeine and an inhibitory effect on the differentiation of the preadipocytes.

Accordingly, according to a first aspect, the subject of the invention is the use of a quinoa extract, as a slimming active agent, and/or as an active agent which makes it possible to inhibit the formation of new fat in the human body, in a composition containing a cosmetical acceptable medium.

The term quinoa denotes, in the present patent application, the quinoa plant (Chenopodium quinoa) or its genetically modified variants. The quinoa extracts used in the invention which is the subject of the present patent application are generally commercially available in the form of liquid extracts as aqueous extracts, glycolic extracts, in which the solvent is, for example, glycol, propylene glycol or butylene glycol, aqueous-glycolic extracts, alcoholic extracts in which the alcohol is, for example, alcoholic ethanol or aqueous-alcoholic extracts. Such extracts generally contain between 0.01% and 10% by mass of dry extract.

The subject of the invention is also a method for the nontherapeutic treatment of the human body intended for slimming it and/or intended for inhibiting the formation of new fat, characterized in that a composition containing a cosmetically acceptable medium and an effective quantity of quinoa extract is applied thereto.

The expression effective quantity denotes, in the context of the present invention, a percentage by mass of dry extract relative to the final composition of between 0.0001% by mass and 2.0% by mass, preferably between 0.0001% by mass and 0.9% by mass.

The subject of the invention is also the use of a quinoa extract, as defined above, for preparing a medicament with lipolytic activity, intended to induce slimming of the human body.

The subject of the invention is also the use of a quinoa extract, as defined above, for preparing a medicament with inhibitory activity on the differentiation of the preadipocytes, intended to prevent the formation of new fat in the human and/or animal body.

In the compositions defined above, the quinoa extract is generally used in a sufficient quantity for there to be between 0.0001% and 2% by mass of composition as dry extract, more particularly between 0.0005% and 0.9% by mass of composition as dry extract and most particularly between 0.001% and 0.6% by mass of composition as dry extract.

As the following experimental study shows, the quinoa extracts used in the cosmetic or therapeutic treatments defined above are characterized, unexpectedly, by a lipolytic activity greater than the compositions of the state of the art and by a capacity to prevent the formation of fat in the human body. They are therefore in general appropriate for treatments for slimming the human body.

The compositions used in said treatments are generally provided in the form of dilute aqueous or aqueous-alcoholic solutions, in the form of simple or multiple emulsions, such as water-in-oil (W/O), oil-in-water (O/W) or water-in-oil-in-water (W/O/W) emulsions in which the oil is of a vegetable or mineral nature, or in powdered form. They may also be dispersed or impregnated onto textile or onto nonwoven materials such as wipes, paper serviettes or clothing.

The compositions used in said treatments are administered to the subject in the conventional forms used in cosmetics and in pharmacy; this includes more particularly topical, oral or parenteral administrations.

In general, the quinoa extracts are combined with many types of adjuvants or active ingredients used in cosmetic formulations, such as fatty substances, organic solvents, thickeners, gelling agents, emollients, antioxidants, opacifiers, stabilizers, foaming agents, perfumes, emulsifiers, which are ionic or nonionic, fillers, sequestrants, chelators, preservatives, chemical screening agents or inorganic screening agents, essential oils, coloring matter, pigments, hydrophilic or lipophilic active agents, humectants, for example glycerin, preservatives, colorants, perfumes, cosmetic active agents, inorganic or organic sunscreens, inorganic fillers such as iron oxides, titanium oxides and talc, synthetic fillers such as nylons and poly(methyl methacrylate) which are crosslinked or otherwise, silicone elastomers, sericites or plant extracts or alternatively lipid vesicles or any other ingredient customarily used in cosmetics.

As examples of oils which may be combined with the quinoa extracts, there may be mentioned mineral oils such as paraffin oil, liquid paraffin, isoparaffins or white mineral oils, oils of animal origin, such as squalene or squalane, vegetable oils, such as sweet almond oil, copra oil, castor oil, jojoba oil, olive oil, rapeseed oil, groundnut oil, sunflower oil, wheat germ oil, maize germ oil, soya bean oil, cottonseed oil, lucerne oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, calophyllum oil, sysymbrium oil, avocado oil, calendula oil; ethoxylated vegetable oils; synthetic oils such as fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, monoglycerides, diglycerides and triglycerides of fatty acids such as glyceryl triheptanoate, alkyl benzoates, poly-alpha-olefins, polyolefins such as polyisobutene, synthetic isoalkanes such as isohexadecane, isododecane, perfluorinated oils and silicone oils. Among the latter, there may be mentioned more particularly dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluorinated groups, cyclic silicones and silicones modified with alkyl groups.

As other fatty substances which may be combined with this active agent, there may be mentioned fatty alcohols or fatty acids.

Among the thickening and/or emulsifying polymers used in the present invention are for example homopolymers or copolymers of acrylic acid or of acrylic acid derivatives, homopolymers or copolymers of acrylamide, homopolymers or copolymers of acrylamide derivatives, homopolymers or copolymers of acrylamidomethylpropane-sulfonic acid, vinyl monomer, trimethylaminoethyl-acrylate chloride, hydrocolloids of plant or biosynthetic origin, for example xanthan gum, karaya gum, carrageenans, alginates; silicates; cellulose and its derivatives; starch and its hydrophilic derivatives; polyurethanes. Among the polymers of the polyelectrolyte type which may be used in the production of a gelled aqueous phase capable of being used in the preparation of W/O, O/W, W/O/W or O/W/O emulsions, or of an aqueous gel containing the quinoa extracts which are the subject of the present invention, there are for example copolymers of acrylic acid and 2-methyl-[(1-oxo-2-propenyl)amino]-1-propane-sulfonic acid (AMPS), copolymers of acrylamide and 2-methyl-[(1-oxo-2-propenyl)amino]-1-propanesulfonic acid, copolymers of 2-methyl-[(1-oxo-2-propenyl)amino]-1-propanesulfonic acid and (2-hydroxyethyl)acrylate, homopolymer of 2-methyl-[(1-oxo-2-propenyl)amino]-1-propanesulfonic acid, homopolymer of acrylic acid, copolymers of acryloylethyltrimethylammonium chloride and acrylamide, copolymers of AMPS and vinylpyrrolidone, copolymers of acrylic acid and alkyl acrylates whose carbon chain comprises between ten and thirty carbon atoms, copolymers of AMPS and alkyl acrylates whose carbon chain comprises between ten and thirty carbon atoms. Such polymers are marketed respectively under the names SIMULGEL™ EG, SEPIGEL™ 305, SIMULGEL™ NS, SIMULGEL™ 800, SIMULGEL™ A, SIMULGEL™ EPG, SIMULGEL™ INS, SIMULGEL™ FL, SEPIGEL™ 501, SEPIGEL™ 502, SEPIPLUS™ 250, SEPIPLUS™ 265, SEPIPLUS™ 400, SEPINOV™ EMT 10, CARBOPOL™, ULTREZ™ 10, ACULYN™, PEMULEN™ TR1, PEMULEN™ TR2, LUVIGEL™ EM, SALCARE™ SC91, SALCARE™ SC92, SALCARE™ SC95, SALCARE™ SC96, FLOCARE™ ET100, FLOCARE™ ET58, HISPAGEL™, NOVEMER™ EC1, ARISTOFLEX™ AVC, ARISTOFLEX™ HBM, RAPITHIX™ A60, RAPITHIX™ A100, COSMEDIA SP and STABILEZE™ 06.

Among the waxes which can be used in the present invention, there may be mentioned for example beeswax; carnauba wax, candelilla wax; ouricury wax; Japan wax; cork fiber or sugarcane wax; paraffin waxes; lignite waxes; microcrystalline waxes; lanolin wax; ozokerite; polyethylene wax; hydrogenated oils; silicone waxes; vegetable waxes; fatty alcohols and fatty acids which are solid at room temperature; glycerides which are solid at room temperature.

Among the emulsifiers which can be used in the present invention, there may be mentioned:
optionally alkoxylated fatty esters of alkyl polyglycosides, and most particularly the ethoxylated esters of methyl polyglucoside such as PEG 120 methyl glucose trioleate and PEG 120 methyl glucose dioleate marketed respectively under the names GLUCAMATE™ LT and GLUTAMATE™ DOE120;
alkoxylated fatty esters such as PEG 150 pentaerythrityl tetrastearate marketed under the name CROTHIX™ DS53, PEG 55 propylene glycol oleate marketed under the name ANTIL™ 141;
carbamates of fatty chain polyalkylene glycols such as PPG 14 laureth isophoryl dicarbamate marketed under the name ELFACOS™ T211, PPG 14 palmeth 60 hexyl dicarbamate marketed under the name ELFACOS™ GT2125;
fatty acids, ethoxylated fatty acids, fatty acid esters of sorbitol, ethoxylated fatty acid esters, polysorbates, polyglycerol esters, ethoxylated fatty alcohols, sucrose esters, alkyl polyglycosides, sulfated and phosphated fatty alcohols or mixtures of alkyl polyglycosides and fatty alcohols described in French patent applications 2 668 080, 2 734 496, 2 756 195, 2 762 317, 2 784 680, 2 784 904, 2 791 565, 2 790 977, 2 807 435, 2 804 432, 2 830 774, 2 830 445, combinations of emulsifying surfactants chosen from alkyl polyglycosides, combinations of alkyl polyglycosides and fatty alcohols, esters of polyglycerols or polyglycols or polyols such as polyhydroxystearates of polyglycols or polyglycerols used in French patent applications 2 852 257, 2 858 554, 2 820 316 and 2 852 258.

As examples of an active ingredient which may be combined with the quinoa extracts there may be mentioned compounds having a lightening or depigmenting action, such as for example arbutin, kojic acid, hydroquinone, ellagic acid, vitamin C, magnesium ascorbyl phosphate, SEPICALM™ MSH, extracts of polyphenols, grape extracts, pine extracts, wine extracts, olive extracts, marc extracts, N-acylated proteins, N-acylated peptides, N-acylated amino acids, such as for example N-lauroylproline, N-linoleyllysine, N-linoleylleucine, N-octanoylglycine, N-undecylenoyl-phenylalanine, N-palmitoylproline, N-acylated partial hydrolyzates of proteins, amino acids, peptides, total hydrolyzates of proteins, partial hydrolyzates of proteins, polyols (for example glycerin or butylene glycol), urea, pyrrolidonecarboxylic acid or derivatives of this acid, glycyrrhetinic acid, alpha-bisabolol, sugars or sugar derivatives, polysaccharides or their derivatives, hydroxy acids, for example lactic acid, vitamins, vitamin derivatives such as retinol, vitamin E and its derivatives, minerals, enzymes, coenzymes such as Coenzyme Q10, hormones or hormone-like substances, soya bean extracts, for example Raffermine™, wheat extracts, for example Tensine™ or Gliadine™, vegetable extracts such as extracts rich in tannins, extracts rich in isoflavones or extracts rich in terpenes, extracts of fresh water or marine algae, essential waxes, bacterial extracts, minerals, lipids in general, lipids such as ceramides or phospholipids, active agents having a slimming action such as caffeine or its derivatives, active agents having an antimicrobial activity or a purifying action in relation to greasy skin such as LIPACIDE™ PVB, LIPACIDE™ UG, active agents having an energizing or stimulating property such as SEPITONIC™ M3 or Physiogenyl™, panthenol and its derivatives such as SEPICAP™ MP, antiaging active agents such as SEPILIFT™ DPHP, LIPACIDE™ PVB, SEPIVINOL™, moisturizing active agents such as SEPICALM™ S, SEPICALM™ VG and LIPACIDE™ DPHP, PROTEOL™ SAV 50, “antiphotoaging” antiaging active agents, active agents protecting the integrity of the dermo-epidermal junction, active agents increasing the synthesis of the components of the extracellular matrix, active agents having a slimming, toning or draining activity such as caffeine, theophylline, cyclic adenosyl monophosphate (cAMP), green tea, sage, ginko biloba, ivy, horse-chestnut, bamboo, ruscus, butcher's broom, centella asiatica, heather, meadowsweet, fucus, rosemary, willow, active agents creating a sensation of “heat” on the skin, such as activators of skin microcirculation (for example nicotinates) or products creating a sensation of “freshness” on the skin (for example menthol and its derivatives).

As sunscreen which may be incorporated into the composition according to the invention, there may be mentioned all those which appear in the amended cosmetics directive 76/768/EEC, annex VII.

The following experimental study illustrates the invention without, however, limiting it.

### A—Evaluation In Vitro of the Lipolytic Activity of the Quinoa Extracts in Cultures of Normal Human Adipocytes by Assaying the Free Fatty Acids

1—Aim and Principle of the Method
The objective of the experiment is to demonstrate, in an in vitro model of isolated human adipocytes, the lipolytic activity of the compounds used. Hydrolysis of the triglycerides to nonesterified fatty acids and glycerol is called lipolysis. Triglycerides are stored in the adipocytes and constitute the fat reserve. For this reserve to diminish, which is the desired aim when slimming products are used, the triglycerides should be hydrolyzed in the form of fatty acids, which acids can be removed from the cell. Hydrolysis of the triglycerides calls into play a hormone-dependent lipase which must be phosphorylated in order to be active. The phosphorylation step uses a kinase and cAMP. An increase in the cAMP content of the adipocytes is necessary for promoting the activity of the lipase and therefore the lipolysis. The method described consists in incubating the products in the presence of human adipocytes in suspension, followed by measurement of the intracellular cAMP level.

2—Experimental Protocol
(i)—Cellular model: The test is carried out using human adipocytes isolated and prepared as a cellular suspension. The adipocytes are isolated from the subcutaneous abdominal adipose tissue recovered during plastic surgery operations (abdominal plastic surgery operations) performed on women. The cells are isolated from fresh tissue. The adipose tissue is isolated and dissociated by the action of a collagenase (SIGMA™, 1 mg/ml, 30 minutes at 37° C., gentle stirring). Collagenase digests the connective tissue present in the adipose tissue. After digestion, the cells are filtered and washed in an appropriate culture medium containing MEM (minimum essential medium) medium free of phenol red, free of glutamine (SIGMA)+2.2 mg/ml of sodium bicarbonate (GIBCO)+50 IU of penicillin (BIOWHITTAKER™)+50 μg/ml of streptomycin (BIOWHITTAKER™)+1% (v/v) of L-glutamine (BIOWHITTAKER™)+0.5% of lipid-free serum albumin (SIGMA™). The adipocyte suspension is used immediately after its preparation.
(ii) Incubation of the products with the adipocytes: The test products are diluted in the adipocyte culture medium. They are incubated with the cells in suspension for two hours at 37° C. (250 μl of product+250 μl of adipocyte suspension). The glycolic quinoa extract, marketed in France by the company PROVITAL, comprises between 0.5% and 1% by mass of dry extract; it is tested at 0.0001% by mass and 0.0005% by mass of dry extract (DE).

3—Evaluation of the Results
(i)—Concentration of free fatty acids: After the incubation, the cell lysis is checked visually by the presence of a lipid layer at the surface of the cellular suspension. The supernatant media are collected. The free fatty acids are assayed by spectrophotometry using a commercial kit (NEFA™ C kit, WAKO), with reference to a fatty acid calibration series. The lipolytic activity of the products is evaluated relative to a control group incubated in the presence of adipocytes and in the absence of product. The reactivity of the adipocytes is systematically checked for the measurement of the lipolytic activity of the reference products, caffeine (1,3,7-trimethyl-xanthine) and theophylline (1,3-dimethyl-2,6-dihydroxy-purine). Five assays are performed for each of the test products.
(ii)—The results of the trials, expressed as an arithmetic mean of the five assays performed for each of the products, are presented in the following table:

| | | | Lipolytic |
|---|---|---|---|
| | | | activity |
| | | Free fatty | (compared |
| | Incubation | acid | with the |
| | concentration | concentration | control = |
| | (weight % DE) | (μmol) | 100) |
| Control | — | 16 ± 3 | 100 |
| Caffeine | 0.0001 | 25 ± 4 | 154 |
| Glycolic | 0.0001 | 20 ± 3 | 126 |
| quinoa | | | |
| extract | | | |
| Theophylline | | | |
| Glycolic | 0.0005 | 23 ± 1 | 141 |
| quinoa | | | |
| extract | | | |

DE: dry extract

These results show that the quinoa extract has a lipolytic activity of the same order of magnitude as that of caffeine, which makes it a candidate as active ingredient of slimming compositions.

### B—Measurement of the Differentiation of the Preadipocytes to Adipocytes, Search for an Inhibitory Effect

1—Principle of the Method
The method described consists in incubating the product in the presence of murine preadipocytes (3T3-L1 line) which, under the influence of an appropriate culture medium, differentiate into mature adipocytes capable of storing triglycerides. The differentiation is evaluated by an enzymatic assay of Glucose-6-phosphate dehydrogenase (G6PDH). This enzyme, which is located within the adipocytes, allows the conversion of Glucose-6-phosphate to Gluconate-6-phosphate in the presence of Nicotinamide Adenine Dinucleotide Phosphate (NAPD) and leads to the formation of Hydrogenated Nicotinamide Adenine Dinucleotide Phosphate (NAPDH) used for the neosynthesis of fatty acids. It is present in the mature adipocytes. The formation of NAPDH is monitored by absorption spectrophotometry at 340 nanometers.

2—Experimental Protocol
(i)—Cellular model: The test is carried out using murine preadipocytes (3T3-L1 cell line) cultured in a monolayer until confluence is obtained. For that, the cells (60×10³ cells/cm²) are inoculated and cultured for 5 days in DMEM medium containing 4.5 g/l of glucose (SIGMA)+10% fetal serum (SIGMA)+50 ID of penicillin (BIOWHITTAKER™)+50 μg/ml of streptomycin (BIOWHITTAKER™) 1% (v/v) of L-glutamine (BIOWHITTAKER™) (medium A). Two days after the cells have reached confluence, they are incubated in a differentiation medium composed of medium A supplemented with 0.1 μM of dexamethasone+0.17 mM of insulin+0.5 mM of isobutylmethylxanthine (medium B). The cells are incubated in this medium for two days and then for two additional days in medium A supplemented with 0.17 M of insulin (medium C). The cells are finally cultured for 5 additional days in medium A. in suspension for two hours at 37° C. (250 μl of product+250 μl of suspension of adipocytes). The glycolic quinoa extract, marketed in France by the company PROVITAL, is tested at 0.0001% by mass and 0.0005% by mass of dry extract (DE).
(ii)—Incubation of the product with the preadipocytes: The glycolic quinoa extract at 0.02% DE is incubated at the time of initiation of the differentiation with medium B. It is then incubated up to the time of the assay.
(iii)—Assay of the G6PDH activity: At the end of the incubation, the cells are washed in Tris-EDTA-HCl buffer (50 mmol.l⁻¹/5 mmol.l⁻¹, pH=7.5) and then lyzed by the action of ultrasound. The cellular lyzate thus obtained is stored in a cool place. The G6PDH activity of the cellular lyzates is measured by adding its substrate, D-glucose 6P (1 mmol) and its cofactor NADP (8 mmol). The kinetics of appearance of NAPDH is measured by spectrophotometry at 340 nm for 2 minutes. The assay is performed against a G6PDH calibration series. The effect of the product on the differentiation of the preadipocytes is evaluated relative to a control group incubated in the presence of the preadipocytes and in the absence of product (differentiated control) and relative to an undifferentiated control group, incubated in the presence of the preadipocytes cultured solely in medium A.

| | Incubation | | |
|---|---|---|---|
| | concentration | G6PDH | |
| | (weight % DE) | (U/ml) | % inhibition |
| Undifferentiated | — | 0.03 ± 0.002 | 100% |
| control | | | |
| Differentiated | 0.0000001 | 0.46 ± 0.03 | 0% |
| control | | | |
| TNFα (tumor | 0.0000001 | 0.005 ± 0.01 | 43 |
| necrosis factor) | | | |
| Glycolic quinoa | 0.02 | 0.35 ± 0.03 | 26 |
| extract | | | |

DE: dry extract

The TNFα tested at 1 ng/ml (that is 10⁻⁷% DE) inhibits by 43% the differentiation of the preadipocytes. The glycolic quinoa extract tested inhibits by 26% the activity of G6PDH which makes it appropriate for use as slimming active ingredient in preventing the formation of fat in the human body.

### C)—Examples of Cosmetic Formulations

In the following examples, the expressed as percentages by weight.

### EXAMPLE 1

### Slimming Body Milk

| | |
|---|---|
| MONTANOV ™ L | 3.00% |
| Phytosqualane | 8.00% |
| Sweet almond oil | 2.00% |
| Water | qs 100% |
| SEPIGEL ™ 501 | 1.50% |
| Glycolic quinoa extract | 3.00% |
| SEPICIDE ™ CI | 0.20% |
| SEPICIDE ™ HB | 0.30% |
| Perfume | 0.30% |

### EXAMPLE 2

### Anti-Sagging Cream (for Use on the Oval of the Face)

| | |
|---|---|
| MONTANOV ™ 202 | 3.50% |
| MONTANOV ™ 14 | 1.00% |
| SEPILIFT ™ DPHP | 1.00% |
| LANOL ™ 1688 | 15.00% |
| Wheat germ oil | 5.00% |
| Water | qsp 100% |
| SIMULGEL ™ EG | 1.30% |
| Glycolic quinoa extract | 2.00% |
| SEPICIDE ™ CI | 0.20% |
| SEPICIDE ™ HB | 0.30% |
| Perfume | 0.10% |

### EXAMPLE 3

### Anti-Plumpness Spray

| | |
|---|---|
| MONTANE ™ 60 | 3.30% |
| MONTANOX ™ 60 | 1.70% |
| Caprylic/capric triglycerides | 6.00% |
| Isohexadecane | 5.00% |
| Magnesium Aluminum Silicate | 1.50% |
| Water | qsp 100% |
| SIMULGEL ™ EG | 1.00% |
| Glycolic quinoa extract | 2.00% |
| Centella asiatica/hydrocotyle | 1.00% |
| extract | |
| SEPICIDE ™ CI | 0.20% |
| SEPICIDE ™ HB | 0.30% |
| Perfume | 0.40% |
| Water | qsp 100% |

### EXAMPLE 4

### Refreshing Slimming Gel

| | |
|---|---|
| SEPIGEL ™ 305 | 3.50% |
| Hydroxyethylcellulose | 1.00% |
| Caffeine | 5.00% |
| Menthol | 0.30% |
| Ethanol | 50.00% |
| Glycolic quinoa extract | 3.00% |
| SEPICIDE ™ LD | 1.00% |
| Perfume | 0.20% |
| Water | qsp 100% |

### EXAMPLE 5

### Slimming Body Fluid

| | |
|---|---|
| SIMULGEL ™ NS | 2.50% |
| Xanthan gum | 0.20% |
| LANOL ™ 99 | 5.00% |
| Glycolic quinoa extract | 2.00% |
| Ginkgo biloba extract | 2.00% |
| Cola extract | 1.00% |
| Ginseng extract | 0.50% |
| SEPICIDE ™ HB | 1.50% |
| Perfume | 0.10% |
| Water | qsp 100% |

### EXAMPLE 6

### Toning Revitalizing Lotion Intended to be Impregnated into Body Wipes

| | |
|---|---|
| Glycolic quinoa extract | 1.50% |
| Glycerin | 5.00% |
| Ethanol | 5.00% |
| Ruscus extract | 3.00% |
| SEPITONIC ™ M3 | 1.00% |
| SEPICIDE ™ CI | 0.20% |
| SEPICIDE ™ HB | 0.30% |
| Water | qsp 100% |

### EXAMPLE 7

### Slimming Shower Gel

| | |
|---|---|
| MONTALINE ™ C40 | 8.00% |
| PROTEOL ™ OAT | 5.00% |
| Sodium lauryl sulfate | 9.00% |
| Glycolic quinoa extract | 3.00% |
| Green tea extract | 1.00% |
| KATHON ™ CG | 0.80% |
| Green colorant | qs |
| Green tea perfume | 1.00% |
| Lactic acid | qs pH = 6.5 |
| Water | qsp 100% |

### EXAMPLE 8

### Biphasic Disinfiltrating Massage

| | |
|---|---|
| Arabica coffee oil | 1.00% |
| LANOL ™ 189 | 20.00% |
| LANOL ™ 99 | 10.00% |
| Borage oil | 2.00% |
| Perfume | 0.10% |
| Glycolic quinoa extract | 3.00% |
| Glycerin | 3.00% |
| Ethanol | 10.00% |
| Blue colorant | qs |
| Water | qsp 100% |

The definitions of the commercial products used in the examples are the following:

SEPILIFT™ DPHP: (INCI name: Dipalmitoyl hydroxyproline), marketed by the company SEPPIC;
SEPICIDE™ CI: Imidazoline urea (preservative), marketed by the company SEPPIC;
SEPICIDE™ HB: Mixture of phenoxyethanol, methylparaben, ethylparaben, propylparaben and butylparaben (preservative), marketed by the company SEPPIC;
SEPICIDE™ LD: Phenoxyethanol marketed by the company SEPPIC;
KATHON™ CG: (INCI name: methyl isothiazolinone/Methyl chloroisothiazolinone);
MONTANE™ 60: Sorbitan stearate;

### MONTANOX™ 60: Polysorbate 60;

SIMULGEL™ EG: Self-reversible invert latex of copolymer such as those described in international publication WO 99/36445 (INCI name: Sodium acrylate/Sodium acryloyldimethyl taurate copolymer and Isohexadecane and Polysorbate 80) marketed by the company SEPPIC;
SIMULGEL™ NS: Self-reversible invert latex of copolymer such as those described in international publication WO 99/36445 (INCI name: hydroxyethylacrylate/Sodium acryloyldimethyl taurate copolymer and squalane and Polysorbate 60) marketed by the company SEPPIC;
SEPIGEL™ 305: Self-reversible invert latex (INCI name: Polyacrylamide/C13-14 Isoparaffin/Laureth-7);
SEPIGEL™ 501: Self-reversible invert latex (INCI name: C13-14 Isoparaffin/Mineral Oil/Sodium polyacrylate-/Polyacrylamide/Polysorbate 85) marketed by the company SEPPIC;
LANOL™ 99: Isononyl isononanoate marketed by the company SEPPIC;
LANOL™ 189: Isostearyl isononanoate;
LANOL™ 1688: Cetearyl ethylhexanoate marketed by the company SEPPIC;
SEPITONIC™ M3: Mixture of magnesium aspartate, copper gluconate and zinc gluconate marketed by the company SEPPIC;
MONTALINE™ C40: Cocamidopropyl betainamide MEA chloride;
PROTEOL™OAT: N-lauroyl-containing oat amino acids;
MONTANOV™ b 14: Myristyl alcohol/Myristyl glucoside;
MONTANOV™ L: Emulsifying agent based on a C14-C22 alcohol and a C12-C20 alkyl polyglucoside such as those described in European Patent Application EP 0 995 487;
MONTANOV™ 202 is an emulsifying agent based on arachidyl alcohol, behenyl alcohol and arachidyl polyglucoside.

1-4. (canceled)

5. A composition for slimming and/or preventing the formation of new fats in the human body, said composition comprising an effective amount of at least one quinoa extract and a cosmetically acceptable medium.

6. The composition of claim 5, wherein the effective amount of the at least one quinoa extract is a percentage by mass of dry extract relative to the final composition of between 0.0001% by mass and 2.0% by mass.

7. The composition of claim 5, wherein the effective amount of the at least one quinoa extract is a percentage by mass of dry extract relative to the final composition of between 0.0001% by mass and 0.9% by mass.

8. The composition of claim 5, wherein the effective amount of the at least one quinoa extract is a percentage by mass of dry extract relative to the final composition of between 0.001% by mass and 0.6% by mass.

9. The composition of claim 5, wherein the composition includes one of more adjuvants or active ingredients selected from fatty substances, organic solvents, thickeners, gelling agents, emollients, antioxidants, opacifiers, stabilizers, foaming agents, perfumes, emulsifiers, which are ionic or nonionic, fillers, sequestrants, chelators, preservatives, chemical screening agents or inorganic screening agents, essential oils, coloring matter, pigments, hydrophilic or lipophilic active agents, humectants, glycerin, preservatives, colorants, perfumes, cosmetic active agents, inorganic or organic sunscreens, inorganic fillers, synthetic fillers, silicone elastomers, sericites or plant extracts or alternatively lipid vesicles or any other ingredient customarily used in cosmetics

10. A method for the non-therapeutic slimming treatment for the human body, wherein said method comprises administering to the human body to be treated a composition comprising a cosmetically acceptable medium and an effective quantity of at least one quinoa extract.

11. The composition of claim 10, wherein the effective amount of the at least one quinoa extract is a percentage by mass of dry extract relative to the final composition of between 0.0001% by mass and 2.0% by mass.

12. The composition of claim 10, wherein the effective amount of the at least one quinoa extract is a percentage by mass of dry extract relative to the final composition of between 0.0001% by mass and 0.9% by mass.

13. The composition of claim 10, wherein the effective amount of the at least one quinoa extract is a percentage by mass of dry extract relative to the final composition of between 0.001% by mass and 0.6% by mass.

14. The method of claim 11, wherein the administration is topical.

15. The method of claim 11, wherein the administration is oral.

16. The method of claim 11, wherein the administration is parenteral.

17. The use of at least one quinoa extract in the manufacture of a medicament with lipolytic activity for the slimming of the human body.

18. The use of at least one quinoa extract in the manufacture of a medicament to prevent the formation of new fat in the human body.
